(19) United States
(12) Patent Application Publication
(10) Pub. No.: US 2024/0293449 A1
(43) Pub. Date: Sep. 5, 2024

(54) COMPOSITION FOR TREATING BONE LOSS

(71) Applicant: KING FAISAL UNIVERSITY, AL-AHSA (SA)

(72) Inventors: BASEM MOHAMED ABDALLAH ABDELSALAM, AL-AHSA (SA); ENAS ALI ABDELKADER, AL-AHSA (SA)

(21) Appl. No.: 18/220,560

(22) Filed: Jul. 11, 2023

Related U.S. Application Data
(62) Division of application No. 18/116,820, filed on Mar. 2, 2023.

(51) Int. Cl. A61K 33/38; A61K 9/51; A61K 36/24; A61P 19/10; B82Y 5/00

(52) U.S. Cl. CPC A61K 33/38; A61K 9/5192; A61K 36/24; A61P 19/10; B82Y 5/00; A61K 2236/333; A61K 2236/53

(57) ABSTRACT

The composition for treating bone loss is a solution of silver nanoparticles in an ethanolic extract of Leptadenia plant leaves. The composition is prepared by extracting dried Leptadenia plant leaf powder in ethanol for 24 hours at room temperature with constant stirring. A sample of the extract is fractionated by column chromatography, and the fractions are tested to determine the most effective fraction for stimulating osteoblast formation from bone marrow-derived mesenchymal stem cells (BMSCs). This fraction is used to synthesize green silver nanoparticles by adding the extract fraction to a solution of silver nitrate with constant stirring for 24 hours at room temperature. A portion of the Leptadenia extract is added to the resulting silver nanoparticles to stimulate osteoblast formation in cultures of BMSCs. The composition may be used to form drug compositions for treating bone loss.

### BACKGROUND

### 1. Field

The disclosure of the present patent application relates to treating bone loss, and particularly to a composition for treating bone loss that includes a synergistic combination of silver nanoparticles and Leptadenia extract.

### 2. Description of the Related Art

Osteoporosis is a systemic bone loss-related disease that characterized by reduced bone mass due to increase bone resorption by osteoclast cells on the expenses of bone formation by osteoblast cells. The bone-forming progenitor osteoblast cells are derived from adult stem cells in bone marrow. Most drug therapy for osteoporosis is based mainly on inhibiting bone resorption (anti-catabolics), rather than enhancing bone formation. Thus, there is a need to develop new drug approach for targeting the stimulation of bone formation.

Nanoparticle-based approaches have been developed and widely used for stem cell regeneration therapy. However, most of these approaches use chemically synthesized nanoparticles to fabricate biocompatible and biodegradable nanoscaffolds/nanofibers for tissue engineering as bone graft alternatives, to use nanoparticles as a drug delivery system, to use nanoparticles for photo-thermal therapy of cancer, and for the formulation of quantum dots for labeling and imaging of the fate of implanted stem cells.

Leptadenia is a genus of shrubs native to Africa, the Arabian peninsula, and regions extending into the Indian peninsula. The genus includes five or six species, including L. pyrotechnica, L. reticulata, L. hastata, etc. that are known to contain a variety of phytochemicals and other useful compounds found to be useful in folk and natural medicines for treatment of various conditions, including rheumatoid and bone pain. However, the use of Leptadenia extracts in combination with noble metal nanoparticles, such as silver nanoparticles, has not been reported.

Thus, a composition for treating bone loss solving the aforementioned problems is desired.

### SUMMARY

The composition for treating bone loss is a solution of silver nanoparticles in an ethanolic extract of Leptadenia plant leaves. The composition is prepared by extracting dried Leptadenia plant leaf powder in ethanol for 24 hours at room temperature with constant stirring. A sample of the extract is fractionated by column chromatography, and the fractions are tested to determine the most effective fraction for stimulating osteoblast formation from bone marrow-derived mesenchymal stem cells (BMSCs). This fraction is used to synthesize green silver nanoparticles by adding the extract fraction to a solution of silver nitrate with constant stirring for 24 hours at room temperature. A portion of the Leptadenia extract is added to the resulting silver nanoparticles to stimulate osteoblast formation in cultures of BMSCs. The composition may be used to form drug compositions for treating bone loss.

These and other features of the present subject matter will become readily apparent upon further review of the following specification and drawings.

### BRIEF DESCRIPTION OF THE DRAWINGS

FIG. 1A is a chart showing cytotoxicity of silver nanoparticles as a function of concentration by MTT assay.

FIG. 1B is a chart showing cytotoxicity of Leptadenia plant extract as a function of concentration by MTT assay.

FIG. 2A is a chart of ALP activity for various samples, including a control, L-AgNPs alone, Leptadenia plant extract alone, and a combination of L-AgNPs and Leptadenia plant extract after 6 days.

FIG. 2B is a chart of matrix mineralization by alizarin red assay for various samples, including a control, L-AgNPs (10 μg/mL) alone, Leptadenia plant extract (100 μg/mL) alone, and a combination of L-AgNPs and Leptadenia plant ( 10/100 μg/mL) extract after 12 days.

FIG. 3A is a chart of ALP activity as a function of dose for L-AgNPs (10 μg/mL) in combination with various concentrations (μg/mL) of Leptadenia plant extract after six days.

FIG. 3B is a chart of matrix mineralization as a function of dose for L-AgNPs (10 μg/mL) in combination with various concentrations (μg/mL) of Leptadenia plant extract after 12 days.

FIG. 4 is a chart of upregulated gene expression by qPCR-based osteogenic gene array analysis for various osteogenic growth factors comparing silver nanoparticles alone to a combination of silver NPs and Leptadenia plant extract.

FIG. 5 is a UV spectrum of the synthesized silver nanoparticles.

FIG. 6 is a TEM micrograph of silver nanoparticles synthesized with L. arborea.

FIG. 7 is an X-ray diffraction (XRD) diffractogram showing the diffraction pattern of silver nanoparticles synthesized with L. arborea.

FIG. 8 is an energy dispersive X-ray (EDX) spectrum o the synthesized silver nanoparticles.

FIG. 9 is a plot of Zeta distribution of the synthesized silver nanoparticles.

FIG. 10A is the FTIR spectrum of L. arborea plant extract.

FIG. 10B is the FTIR spectrum of silver nanoparticles synthesized with L. arborea plant extract.

Similar reference characters denote corresponding features consistently throughout the attached drawings.

### DETAILED DESCRIPTION OF THE PREFERRED EMBODIMENTS

The composition for treating bone loss is a solution of silver nanoparticles in an ethanolic extract of Leptadenia plant leaves. The composition is prepared by extracting dried Leptadenia plant leaf powder in ethanol for 24 hours at room temperature with constant stirring. A sample of the extract is fractionated by column chromatography, and the fractions are tested to determine the most effective fraction for stimulating osteoblast formation from bone marrow-derived mesenchymal stem cells (BMSCs). This fraction is used to synthesize green silver nanoparticles by adding the extract fraction to a solution of silver nitrate with constant stirring for 24 hours at room temperature. A portion of the Leptadenia extract is added to the resulting silver nanoparticles to stimulate osteoblast formation in cultures of BMSCs. The composition may be used to form drug compositions for treating bone loss.

The composition for treating bone loss will be explained in the following examples. Materials and test methods in the examples include the following.

Primary mouse bone marrow derived-mesenchymal stem cells (BMSCs) were isolated from 8-week-old male C57BL/6J mice. Cells were cultured in RPMI-1640 medium supplemented with 12% FBS (Thermo Fisher Scientific GmbH, Germany), 12 μM L-glutamine (Thermo Fisher Scientific GmbH) and 1% penicillin/streptomycin (P/S) (Thermo Fisher Scientific GmbH). After 24 h, non-adherent cells were removed and cultured in 60 cm2. Medium was changed every 3-4 days and cells were washed and regularly sub-cultured.

Human bone marrow derived-mesenchymal stem cells were purchased from Cell Applications Inc. (San Diego, CA). Cells were cultured in Dulbecco's modified Eagle medium (DMEM)/low glucose (Sigma-Aldrich GmbH, Germany) containing 10% FBS (Thermo Fisher Scientific GmbH) and 1% penicillin/streptomycin according to the manufacturer's instruction. Medium was changed every 2-3 days.

Cell toxicity of butein was determined by measuring cell viability using MTT cell proliferation assay kit (Sigma-Aldrich) according to the manufacturer's instruction kit. Cells were incubated with MTT solution to metabolize to formazan and absorbance was measured at a wavelength of 550 nm for MTT assay. Values were represented as fold change of control non-treated cells.

Osteoblast differentiation was performed by inducing the cells with osteogenic induction medium (OIM) in α-minimum essential medium (α-MEM; Thermo Fisher Scientific GmbH) supplemented with 10% FBS, 10 mM β-glycerol-phosphate, 100 U/mL of penicillin, 100 mg/mL of streptomycin (Sigma-Aldrich), and 50 mg/mL of vitamin C (Sigma-Aldrich). Cells were either induced with L-AgNPs (10 μg/mL) alone, Leptadenia plant extract (100 μg/mL), or a combination of L-AgNPs and Leptadenia plant extract ( 10/100 μg/mL) for 12 days. The particular species used in these experiments is Leptadenia arborea. Medium was changed every third day during osteogenesis.

Alkaline phosphatase (ALP) activity was tested as follows. Cells were induced with OIM in 96 well plate. ALP activity was determined by incubating the cells with 1 mg/mL of P-nitro phenyl phosphate in 50 mM NaHCO₃ and 1 mM MgCl₂ buffer (p11 9.6) at 37° C. for 20 min. Absorbance was measured at 405 nm. Cell viability was determined using the CellTiter-Blue® cell viability assay according to the manufacturer's instructions in the kit. The value of ALP activity was normalized to the value of cell viability and represented as fold-change over control. Each sample was measured in 6 biological replicates.

Alkaline phosphatase staining was performed as follows. Osteogenic cells were fixed with acetone/citrate buffer pH 4.2 (1.5:1) for 5 min at room temperature. Cells were stained with Napthol-AS-TR-phosphate solution (Sigma-Aldrich) for 1 h at room temperature. The staining solution consists of 1:1 v/v Napthol-AS-TR-phosphate solution (Napthol-AS-TR-phosphate diluted 1:5 in H2O) and Fast Red TR solution (Sigma-Aldrich ApS) (diluted 1:1.2 in 0.1 M Tris buffer, pH 9.0).

Alizarin red S staining and quantification was performed as follows. Cells induced to osteogenic lineage were fixed with 70% ice-cold ethanol for 1 hour at 20° C. and stained with Alizarin red (40 mM, p1=4; Sigma-Aldrich) for 10 min at room temperature. For quantification of calcium deposition, AR-S was eluted with 10% cetylpyridinium chloride (Sigma-Aldrich ApS) for 1 hour at room temperature and the absorbance was measured at 570 nm. Values were normalized to cell number and presented as fold-change over control in non-induced cells.

Osteogenic QPCR array analysis was performed as follows. Mouse bone marrow stem cells (mBMSCs) were induced to osteoblast differentiation in the presence or the absence of butein. Total RNA was extracted after 6 days of induction. Mouse osteogenic RT² Profiler™ PCR array, containing 84 osteoblast-related genes (Qiagen Nordic) was performed using SYBR® Green qPCR method on Applied Biosystems 7500 real-time PCR system. Upregulated genes by butein were represented as fold-change over control (>2 fold, p<0.005) after normalization to reference genes.

### Example 1

### Preparation of Leptadenia Plant Extract

100 g of dried plant leaves powder are suspended in 300 mL of 95% ethanol for 24 h at 37° C. A sample from ethanolic plant extract are applied to a Sephadex LH-20 column chromatography. After comparison with TLC (Thin layer chromatography), several fractions are obtained. Plant extract fractions are screened for the induction of osteoblast differentiation of bone marrow-derived mesenchymal stem cells. The following assays are used: quantitative alkaline phosphatase activity and quantitative Alizarin red matrix mineralization assay. After comparison between different fractions, the plant extract fraction with the highest osteoblast differentiation activity are selected (named OB-fraction) for further experiment to be used for the bio-fabrication of AgNPs. Gas chromatography-mass spectrometry is used for the identification of the compounds in the selected OB-fraction. The OB-fraction is filtered and evaporated by a rotary vacuum evaporator at 40° C., and filtered through Whatman No. 1 filter paper and stored at 4° C.

### Example 2

### Synthesis of Green Silver Nanoparticles (L-AgNPs)

A total of 220 mL of plant genus Leptadenia extract was added to 110 mL of 10 mM silver nitrate (AgNO₃) solution. The solution was stirred for 24 h at room temperature. AgNPs were collected by centrifugation at 12,000 rpm for 15 min at 4° C. The pellet was re-dispersed in water, centrifuged, and lyophilizated (freeze-dried) to obtain L-AgNPs powder. L-AgNPs were characterized by imaging (transmission electron microscopy (TEM), UV-VIS spectroscopy, zeta potential, X-ray diffraction (XRD), Energy dispersive x-ray analysis (EDX), and Fourier transform infrared spectroscopy (FTIR).

### Example 3

### Characterization of Synthesized Silver Nanoparticles

The green silver nanoparticles (L-AgNPs) were characterized by several tests.

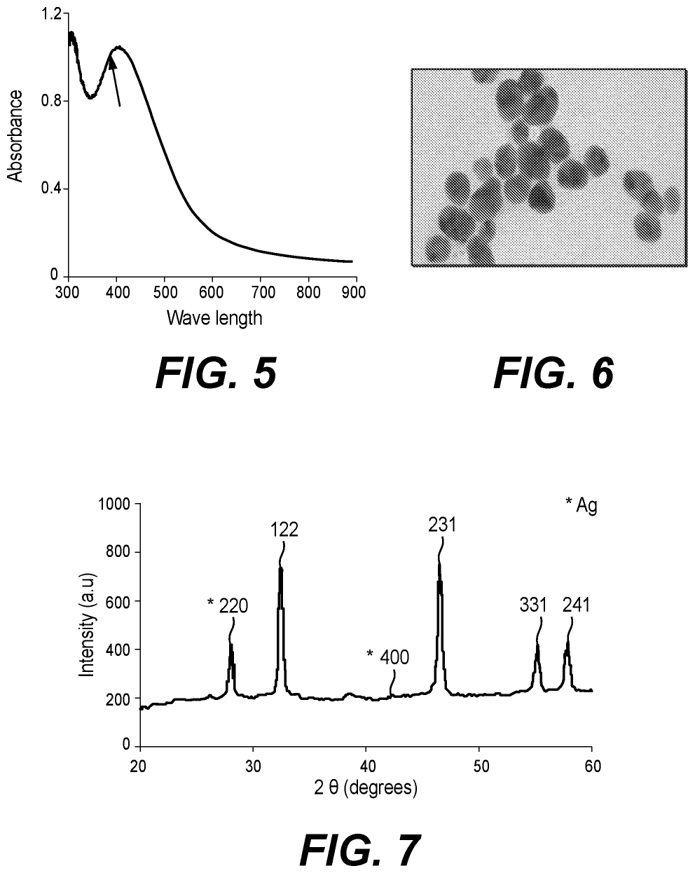

FIG. 5 shows the UV spectrum of silver ions reduced to silver nanoparticles. The distinctive Surface plasma resonance (SPR) of silver nanoparticles ranges between 300 nm to about 600 nm, and exact peaks is at 431 nm.

FIG. 6 is a transmission electron microscopic (TEM) image of green synthesized AgNPs using L. arborea. The AgNPs size observed by TEM is approximately 20 nm in size.

FIG. 7 is an X-ray diffraction (XRD) diffractogram of the biosynthesized AgNPs by using L. arborea extract with distinctive peaks observed in the XRD pattern at 2θ=28.09° marked at (220). A number of Bragg reflections corresponding to the (220) sets of lattice planes are detected, which might be indexed depending on the face-centered crystal structure of silver. Therefore, the XRD pattern obviously displays that the AgNPs are crystalline in nature.

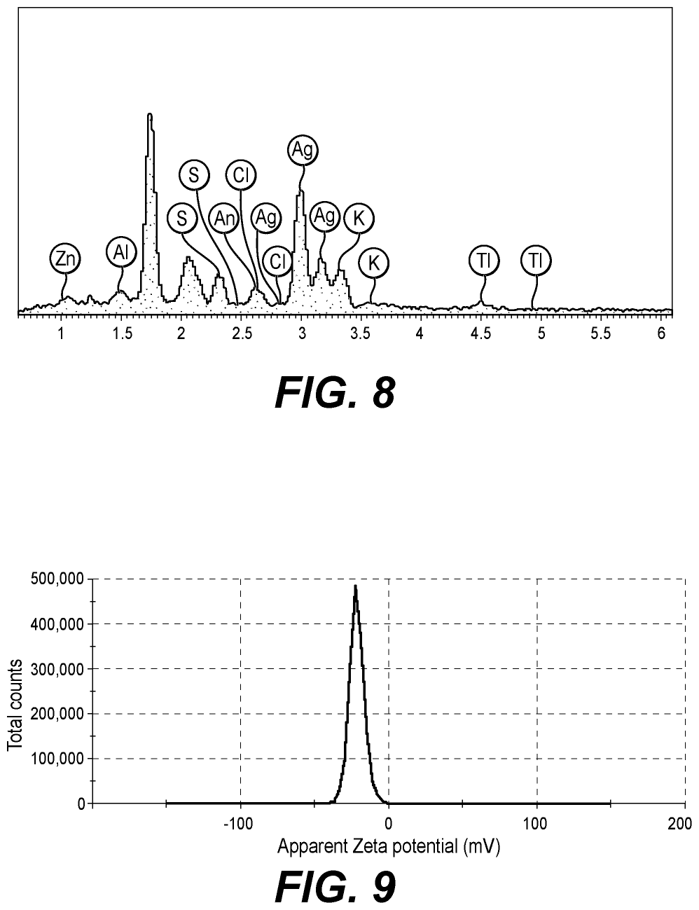

FIG. 8 shows the Energy dispersive x-ray (EDX) analysis of green AgNPs with an intense absorption peak at about 3 keV, which is typical of silver nanoparticles. The silver content of the AgNPs was approximately 68.27%, demonstrating that silver was the dominant element.

FIG. 9 demonstrates the Zeta potential distribution of the AgNPs. Green AgNPs display a significant negative surface charge (−22.3 mV), which prevents particle aggregation because of the repulsive forces and suggests a good stability of nanoparticles.

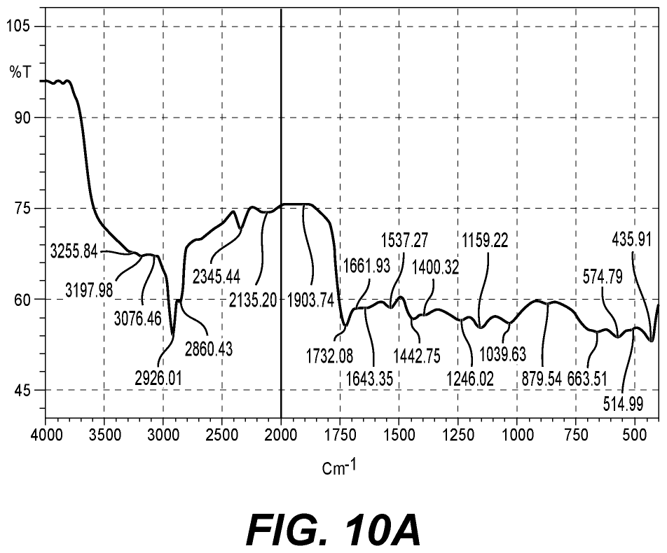

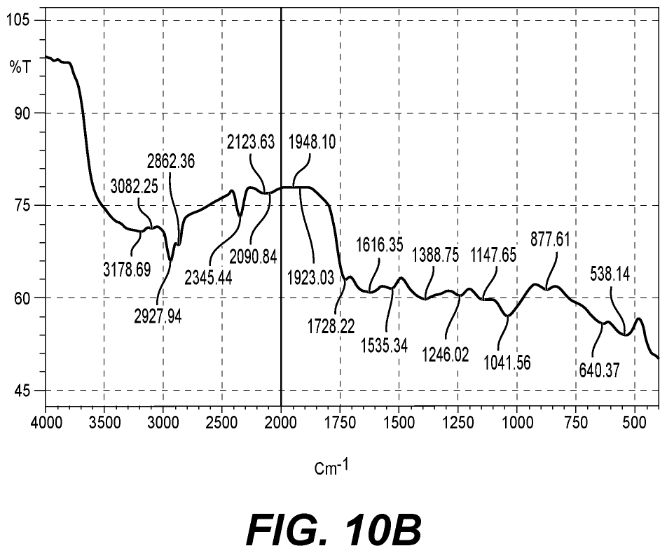

FTIR analysis of both the plant extract alone and of the silver nanoparticles in combination with the plant extract was performed to discover the differences in functional groups, which might affect the activity of the combination. FIG. 10A shows the FTIR spectrum of the plant extract alone and FIG. 10B shows the FTIR spectrum of the plant extract in combination with the silver nanoparticles. In FIG. 10A, the absorption peaks at 3255.84, 3197.98, and 3076.46 cm⁻¹ are associated with amide and hydroxyl stretching vibrations, respectively. In FIG. 10B, the peak for amide stretching was obtained at 3178.60 cm⁻¹. The peaks at 3082.25 and 2862.36 cm⁻¹ are associated with symmetric CH₂ stretching. The peak for the carbonyl group was obtained at 1728.22 cm⁻¹. The carbonyl groups proved the presence of flavanones that are adsorbed on the surface of the nanoparticles.

### Example 4

### Effect of L-AgNPs and Leptadenia Plant Extract on Cell Viability of BMSCs

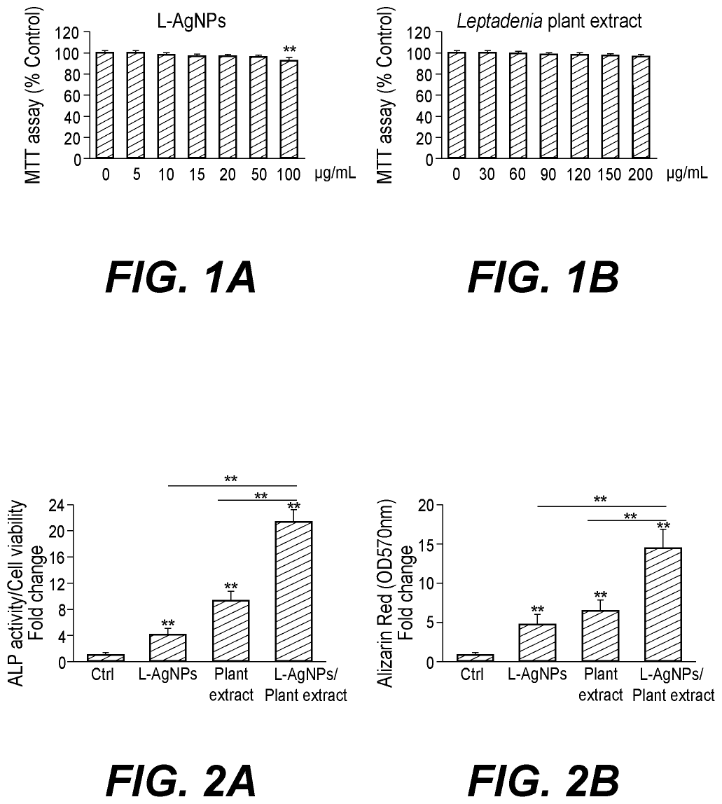

FIGS. 1A and 1B show no cytotoxicity of using silver nanoparticles (L-AgNPs) alone or in a combination with Leptadenia plant extract at different concentrations on bone marrow stem cells (BMSCs). L-AgNPs showed some cytotoxicity at 200 μg/mL. The cytotoxicity of green synthesized L-AgNPs (FIG. 1A) and Leptadenia plant extract (FIG. 1B) on cultured BMSCs at different concentrations are shown. Cell viability was determined by MTT assay. Cells were either non-treated (0) or treated with different concentrations of L-AgNPs or Leptadenia plant extract for 4 days. Values were represented as fold-change over control in non-induced cells. Values are mean±SD of three independent experiments, (*p<0.05, **p<0.005).

First, the L-AgNPs were examined for cytotoxicity on BMSCs to determine the optimal concentration for the osteoblast differentiation process. BMSCs were cultured in 3D as suspended cells in a rotary cell culture system in the presence of cell culture DMEM medium supplemented with L-AgNPs and OB-fraction Leptadenia extract for 2 days. Cells were then transferred to an adherent cell culture flask and cultured as adherent monolayer cells in α-MEM medium (80% v/v); supplemented with L-AgNPs, OB-fraction Leptadenia extract (20% v/v), 10% FBS, 10 mM β-glycerol-phosphate, and 50 mg/ml of vitamin C. Cells were cultured in 37° C. incubator for 10 days with medium change every 3 days.

To assess the osteoblast differentiation of BMSCs, the following assays were performed: quantitative alkaline phosphatase activity was performed after 5 days of induction; quantitative Alizarin red staining for matrix mineralization was performed after 10 days of induction.

### Example 5

### Synergistic Effect of a Combination of L-AgNPs and Leptadenia Plant Extract on Osteoblast Differentiation

FIGS. 2A and 2B show the synergistic effect of a combination of L-AgNPs and Leptadenia plant extract on osteoblast differentiation. BMSCs were treated for up to 12 days with L-AgNPs (10 μg/mL) alone or in a combination with Leptadenia plant extract (100 μg/mL). Osteoblast differentiation was assessed with quantification of ALP activity and matrix mineralization. L-AgNPs synergized the stimulatory effect of Leptadenia plant extract on osteoblast differentiation of BMSCs.

The synergistic effect of a combination of L-AgNPs and plant extract on stimulating osteoblast differentiation of BMSCs as measured by quantification of ALP activity (FIG. 2A) and matrix mineralization with Alizarin red staining after 6 and 12 days of induction (FIG. 2B) are shown, respectively. Cells were either non-induced (Ctrl, control), or induced with L-AgNPs (10 μg/mL), Leptadenia plant extract (100 μg/mL), or a combination of L-AgNPs and Leptadenia plant extract ( 10/100 μg/mL). Values were represented in FIGS. 2A and 2B as fold-change over control in non-induced cells. Values are mean±SD of three independent experiments, (**p<0.005).

### Example 6

### Dose-Dependent Stimulatory Effect of a Combination of L-AgNPs and Leptadenia Plant Extract on Osteoblast Differentiation

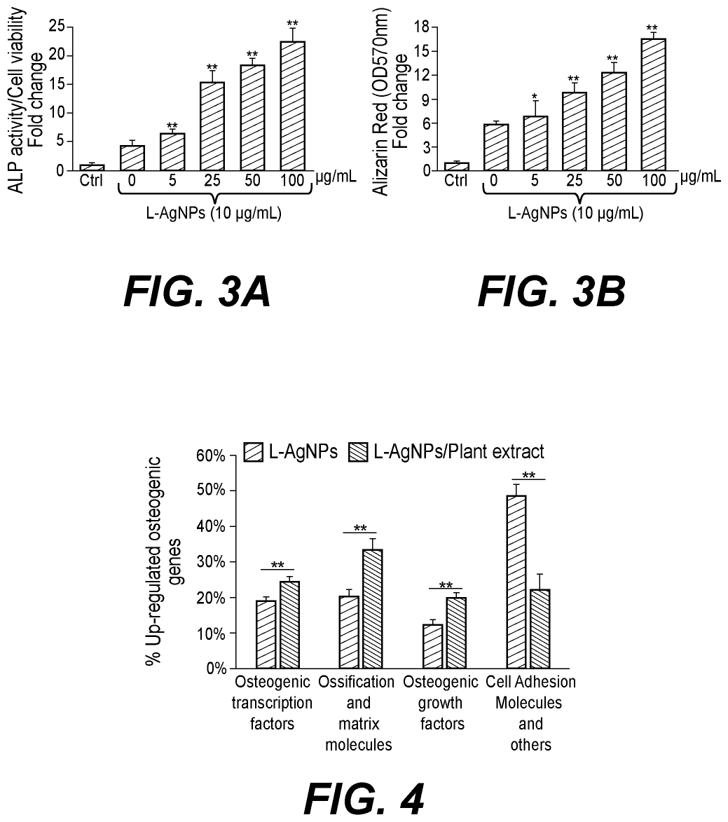

FIGS. 3A and 3B show the synergistic effect of L-AgNPs on the stimulatory effect of plant extract on the osteoblast differentiation of BMSCs in dose-dependent manner. Osteoblast differentiation was assessed with quantification of ALP activity (FIG. 3A) and matrix mineralization (FIG. 3B), respectively. L-AgNPs synergized the stimulatory effect of Leptadenia plant extract on osteoblast differentiation of BMSCs.

Human bone marrow stem cells (hBMSCs) were either non-induced (Ctrl, control), or induced with L-AgNPs (10 fig/mL) in the absence or the presence of different concentrations of Leptadenia plant extract (100 μg/mL). FIG. 3A shows Quantification by ALP activity measured after 6 days of treatment. FIG. 3B shows Quantitative Alizarin red staining for matrix mineralization measured after 12 days of treatment. Values were represented as fold-change over control in non-induced cells. Values are mean #SD of three independent experiments, (**p<0.005 compared to cells treated with L-AgNPs (10 [μg/mL) alone).

### Example 7

### Synergistic Effect of a Combination of L-AgNPs and Leptadenia Plant Extract on Stimulating the Osteoblast-Related Genes Expression in BMSCs

As shown in FIG. 4, L-AgNPs synergized the stimulatory effect of Leptadenia plant extract on upregulating the gene expression of osteoblast-related genes in BMSCs. Upregulated osteoblastic gene expression of L-AgNPs/Plant extract in combination versus L-AgNPs alone in BMSCs was measured by qPCR-based osteogenic gene array analysis. Upregulated gene expression was categorized according to osteogenic function. Values are mean+SD of three independent experiments, (**p<0.005 compared to differentiated cells with L-AgNPs).

The above-described composition or treating bone loss may be used as a pharmaceutical, or used to develop a pharmaceutical, as an emulsion or in liquid or powder form, with or without the addition of excipients, such as binders and fillers, and may be formulated as a capsule or tablet for oral administration or may be administered intravenously by injection or infusion. It is anticipated that the dosage may be determined without undue experimentation and adjusted as needed by routine monitoring, such as testing bone density, to treat and counteract bone porosity in osteoporosis and similar conditions.

It is to be understood that the composition for treating bone loss is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A composition for treating bone loss, comprising a mixture of silver nanoparticles and an extract of a plant of the genus Leptadenia, wherein the mixture comprises: 10 μg/mL silver nanoparticles; and up to 100 μg/mL of the extract of the plant of the genus Leptadenia.

2. The composition for treating bone loss according to claim 1, wherein the extract of the plant of the genus Leptadenia is an extract in ethanol as an extraction solvent.

3. The composition for treating bone loss according to claim 1, wherein the silver nanoparticles are prepared by precipitation in an extract of the plant of the genus Leptadenia.

4. (canceled)

5. The composition for treating bone loss according to claim 1, wherein the extract of the plant of the genus Leptadenia comprises a fraction of the extract showing the greatest to generate osteoblasts in bone marrow stem cells as determined by quantitative alkaline phosphatase activity assay.

6. The composition for treating bone loss according to claim 1, wherein the extract of the plant of the genus Leptadenia comprises a fraction of the extract showing the greatest to generate osteoblasts in bone marrow stem cells as determined by quantitative Alizarin red matrix mineralization assay.

7-13. (canceled)
